(19) United States
(12) Patent Application Publication
(10) Pub. No.: US 2009/0197942 A1
(43) Pub. Date: Aug. 6, 2009

(54) Method For Producing Polyphenol-Rich Composition

(75) Inventors: Mihoko Kurokawa, Fukuoka (JP); Naonori Uchida, Fukuoka (JP); Seiji Imazumi, Fukuoka (JP); Fumio Eguchi, Gunma (JP)

Correspondence Address: RANKIN, HILL & CLARK LLP, 925 EUCLID AVENUE, SUITE 700, CLEVELAND, OH, 44115-1405, US

(73) Assignee: KYUSAI CO., LTD., Fukuoka-shi, Fukuoka (JP)

(21) Appl. No.: 12/301,066

(22) PCT Filed: May 22, 2007
(86) PCT No.: PCT/JP2007/060455

§ 371 Date: Nov. 17, 2008

(30) Foreign Application Priority Data
May 23, 2006 (JP) 2006-142385

(51) Int. Cl. A61K 31/352; C08B 1/00; C08B 31/00; A23G 3/00; A23L 1/05

(52) U.S. Cl. 514/456; 536/56; 536/102; 536/123.1; 426/658; 426/661

(57) ABSTRACT

An object of the present invention is to provide a method for producing polyphenol-containing compositions and purifying polyphenols safely and simply. By contacting a polyphenol-containing solution to a specific polysaccharide to support polyphenols with the polysaccharide, a polyphenol-containing composition usable as an additive for foods and drinks can be obtained. In addition, polyphenol can be purified by contacting polysaccharide-supported polyphenols with solvent to elute polyphenols into the solvent and then powderizing the polyphenols after removing the solvent.

### RELATED APPLICATIONS

This application claims the priority of Japanese Patent Application No. 2006-142385 filed on May 23, 2006 which is incorporated herein by reference.

### FIELD OF THE INVENTION

The invention relates to methods for producing polyphenol-rich compositions and purifying polyphenols, and in particular, to compositions for inhibiting hypertension, obtained by adding arginine to quercetin which is a type of polyphenol.

### BACKGROUND OF THE INVENTION

Polyphenol is a generic term for compounds having two or more phenolic hydroxyl groups within the same molecule. Most types of polyphenols are widely distributed in plants. Many flavonoids, which are typical polyphenols, have biological regulation functions such as anti-oxidative, anti-mutagenic, anti-cancer, anti-diabetic, and anti-allergic activities and may exhibit effects for inhibiting hypertension. In particular, it has been reported that quercetin, a type of flavonoid, has an excellent effect for inhibiting hypertension.

In recent years, the beneficial effects of polyphenols have drawn attention along with growing concerns for improved health. Polyphenols are now used in various fields such as food, cosmetics, drugs and so on. When polyphenol is used as an additive or a starting material in such fields, the polyphenol, which is a specific fraction, needs to be separated from a raw material containing the polyphenol. A conventional method for separating polyphenols involves a technique for selectively adsorbing polyphenols to adsorbent agents. For example, a method for separating polyphenols by contacting a polyphenol-containing solution with a styrenic synthetic adsorbent and adsorbing polyphenols to the adsorbent is described in Patent Literature 1. And, a method for separating polyphenols by blending an extraction liquid from onion skins with proteins to prepare a solution containing polyphenolic compounds, contacting the solution with wood powder to adsorb polyphenols to the wood powder, and eluting polyphenols into a solvent have been reported in Patent Literature 2.

Additionally, as a technique for using edible substances as adsorbent agents, a method for liberating quercetin which is a type of flavonoid, by forming a complex of proteins and quercetin has been reported in Patent Literature 3.

### Patent Literature 1: Japanese Unexamined Patent Publication 2002-335911

### Patent Literature 2: Japanese Unexamined Patent Publication H7-238078

### Patent Literature 3: Japanese Unexamined Patent Publication 2005-289850.

However, in adsorption purification methods of polyphenols such as Patent Literatures 1 and 2, a utilization of adsorbent agents which adsorb polyphenols has not been focused on. That is, in these conventional adsorption purification methods, using edible substances as adsorbent agents or utilizing adsorbent agents which adsorb polyphenols as polyphenol-rich food materials has not been considered.

In the technique of Patent Literature 3 for using an edible protein substance as an adsorbent agent, a safety concern arises because there is a risk of adsorbing residual pesticides in addition to polyphenols during the operation of adsorbing polyphenols to the protein.

Furthermore, many of these concerns are particularly significant for quercetin which has received attention in recent years. Quercetin requires an excellent extraction and separation method in terms of recovery rate and degree of purity in the recovered product.

### DISCLOSURE OF THE INVENTION

### Problem to be Solved by the Invention

The present invention is provided in view of the previously noted problems and objects thereof. The invention provides a method for producing polyphenol-rich compositions, purifying polyphenols in a safe and efficient manner, and certain polyphenol-rich compositions.

### Means to Solve the Problem

The present inventors have diligently studied to achieve the above-described objects. As a result, the present inventors have found that polysaccharide-supported polyphenol usable as food materials, can be obtained by supporting polyphenols, which are included in a polyphenol-containing solution, with one or more specific polysaccharides.

In addition, the present inventors have also found that it is possible to purify polyphenols from the polysaccharide-supported polyphenols and that a composition containing arginine and quercetin which is a type of polyphenol, has an excellent effect for inhibiting hypertension, thus leading to completion of the present invention.

Specifically, the present invention provides a method for producing a polyphenol-rich composition. The method comprises contacting a polyphenol-containing solution with one or more polysaccharides selected from a group consisting of cellulose, starch, chitin, chitosan, and combinations thereof. The method also comprises supporting the polyphenols with the polysaccharide(s) to produce a polyphenol-rich composition. In addition, in the method for producing the polyphenol-rich composition, it is preferred that the cellulose is crystalline cellulose, carboxymethylcellulose, microfibrillar cellulose, powder cellulose, and combinations thereof.

In addition, when producing the polyphenol-rich composition, it is preferred that the polyphenol is quercetin. The present invention method enables the production of quercetin in high concentrations. In addition, in the present invention method for producing a polyphenol-rich composition, it is preferred that the polyphenol-containing solution is a solution containing quercetin extracted from onion skins with water or a solution of 40 percent or less of ethanol, methanol or acetone.

In addition, the present invention provides foods and drinks containing polysaccharide-supported polyphenols. In the foods and drinks, it is preferred that the polysaccharide to support polyphenols is starch.

The present invention also provides a method for purifying polyphenols comprising contacting polysaccharide-supported polyphenols with solvent to elute polyphenols into the solvent. The method also comprises powderizing the polyphenols after removing the solvent.

In the method for purifying polyphenols, it is preferred that the polyphenol is quercetin.

The present invention also provides a composition for inhibiting hypertension comprising quercetin and arginine.

### EFFECT OF THE INVENTION

In accordance with the present invention, polyphenols can be purified safely and simply from polyphenol-containing vegetative materials, and highly concentrated polyphenols can be effectively consumed by adding polyphenols together with adsorbent agents to foods and drinks as food materials.

Furthermore, if quercetin is applied as polyphenols, addition of arginine makes it possible to obtain a composition having an excellent effect for inhibiting hypertension.

### BRIEF DESCRIPTION OF THE DRAWINGS

FIG. 1 is a flowchart of a process in accordance with the present invention.

FIG. 2 is a graph showing changes in blood pressures of SHR rats described in Example 2.

FIG. 3 is a graph showing changes in blood pressures of WKY rats described in Example 2.

### BEST MODE FOR CARRYING OUT THE INVENTION

Hereinafter, the present invention is explained in more details.

A wide array of polyphenols can be used in the present invention. Examples of such polyphenols include, but are not limited to, those of the phenylcarboxylic acid series, lignan series, curcumin series, coumalin series and flavonoids series; and preferably polyphenols which are classified as flavonoids.

Examples of the flavonoid polyphenols include flavones such as apigenin, chrysin and luteolin; flavonols such as galangin, quercetin, rutin, kaempferol and myricetin; isoflavones such as daidzein, daidzin and genistein; flavanols such as catechin, epicatechin, epigallocatechin, epicatechin gallate, epigallocatechin gallate and theaflavin; flavanones such as naringenin, naringin, hesperetin and hesperidin; anthocyanidins such as anthocyanidin, anthocyanin, cyanidin, cyanine, proanthocyanidin, ploanthocyanin, delphinidin, delphinine, pelargonidin and pelargonin; and glycosides thereof. In the present invention, quercetin, catechin, epicatechin, epigallocatechin, epicatechin gallate, epigallocatechin gallate or proanthocyanidin is preferably used, and more preferably quercetin.

The polyphenol-containing solution in the present invention may be any solution as long as the solution contains at least one polyphenol. Examples of the polyphenol-containing solution include, but are not limited to, an extract of materials containing polyphenols such as plants, residues such as juice, mixtures, soup and strained lees derived from processing polyphenol-containing materials, and so on. Because the types and contents of polyphenols vary with each material, the materials to be used may be appropriately selected according to desired polyphenols. In the present invention, a solution containing quercetin extracted from onion skins is preferably used.

In accordance with the present invention, when using plant extract in a polyphenol-containing solution, the particular extraction method used is not particularly restricted, and commonly applied extraction methods may be used. For example, in one embodiment, extract without impurities can be obtained by immersing vegetative materials into a solvent, stirring extracting ingredients and heating appropriately as needed, and filtering the solution after the extraction. The solvent for extraction can be selected from organic solvents such as water, ethanol, propylene glycol, 1,3-butylene glycol, methanol, hexane, acetone, diethyl ether, dioxane and ethyl acetate. In a preferred embodiment, if water or 40 percent or less of ethanol, methanol or acetone solution is used, the same solution can be used from the above-mentioned extraction process for the following process for supporting polyphenols with polysaccharide(s), which is preferable in terms of efficiency of implementation. In order to promote the amount of polyphenols extracted from vegetative materials, 30 to 80% of ethanol solution is preferably used. If using highly concentrated organic solutions in the extraction process, however, an operation of removing the solvent or diluting the solvent with water may be required prior to the process for supporting polyphenols.

In the present invention, cellulose, starch, chitin, or chitosan is preferably used as the polysaccharide to support polyphenols. As cellulose, crystalline cellulose, carboxymethylcellulose, microfibrillar cellulose, or powder cellulose is more preferably used.

The process for supporting polyphenols is conducted by contacting a polyphenol-containing solution with the one or more selected polysaccharide(s) and selectively supporting the polyphenols with the polysaccharide(s). The amount of the polysaccharide in the solvent or solution is not particularly restricted, and it may be set appropriately depending on the amount of desired extract, a concentration of polyphenols to be supported, and the particular application. Water or 40 percent or less of ethanol, methanol or acetone solution is preferably used as solvent in the process for supporting polyphenols. If a concentration of the organic solvent exceeds 40 percent, the amount of polyphenol supported with the polysaccharide decreases.

The polysaccharide, with which one or more polyphenols are supported, can be added to foods and drinks as polysaccharide-supported polyphenols if the solvent is removed by drying, reducing the pressure or other similar techniques. Furthermore, the polysaccharide-supported polyphenols can itself be consumed as foods. When consuming polysaccharide-supported polyphenols, a digestible polysaccharide is more preferably used for supporting the polyphenols, in view of the increased absorption efficiency of supported polyphenols.

The drinks and foods, to which the polysaccharide-supported polyphenols of the present invention is added, include, but are not limited to, common foods such as snacks, noodles, dairy products, processed aquatic products, processed livestock products, processed vegetable and fruit products, fermented foods, condiments, miso soup, soups, and beverages; health food products; nutraceutical products; buccals such as gums, candies and tablets; instant food products and the like.

The amount of the polysaccharide-supported polyphenols which is added to the drinks and foods in accordance with the present invention may be appropriately determined depending on the foods and drinks to be manufactured. And, any of the drinks and foods can be manufactured in conventional manners.

Regarding the polysaccharide-supported polyphenols, polyphenols can be purified by contacting the polysaccharide-supported polyphenols with solvent to elute polyphenols supported with the polysaccharide into the solvent. Regarding the solvent used for eluting polyphenols, organic solvents such as water, ethanol, propylene glycol, 1,3-butylene glycol, methanol, hexane, acetone, diethyl ether, dioxane and ethyl acetate can be used alone or in combination thereof. Water, ethanol, methanol and acetic ether are preferably used; and more preferably 40 to 80% of ethanol solution is used.

After eluting one or more polyphenols into the solvent, if the polysaccharide is removed with filtering and the solvent is removed by concentrating, purified polyphenols can be obtained as residues.

The purified polyphenols can be powderized by drying by such methods as freeze drying or spray drying.

The polyphenols purified by the preferred embodiment method of the present invention can be used in numerous applications, such as applications in which polyphenols are generally used, such as foods, cosmetics and drugs.

A composition containing arginine and quercetin which is a type of polyphenol, achieves an excellent effect for inhibiting hypertension. It is known that quercetin exhibits an inhibitory effect for hypertension. However, the present inventors found that the effect for inhibiting hypertension by quercetin is significantly enhanced by adding arginine, which is a type of amino acid.

An effect for inhibiting hypertension can be achieved by consuming 0.7 or more mg/day/kg of quercetin. Considering both the effect and dosage, it is preferred that quercetin and arginine are added to a composition in a ratio of 10 to 23:1.

Purified quercetin is preferably used in a composition for inhibiting hypertension in accordance with the present invention. However, quercetin-containing compound(s) may be also used so far as the compound(s) contains an effective amount of quercetin. That is, in view of the preferred methods of the present invention, any of the following can be used to administer quercetin: a quercetin-containing solution prior to contacting with polysaccharide, a polysaccharide-supported quercetin obtained by contacting the solution with polysaccharide, quercetin purified from the polysaccharide-supported quercetin, or compositions formed by blending any of the foregoing can be used.

Likewise, purified arginine or an arginine-containing compound can be used in the present invention. Furthermore, either arginine obtained by conventional purification methods or commercially available arginine may be used.

The composition for inhibiting hypertension containing the above-mentioned quercetin and arginine can be blended in foods, drugs, cosmetics and so on and may be used in combination with ingredients or additives which are generally used in such products. The blending amount of the composition is not restricted in particular.

The present invention will now be described in more detail with reference to the noted examples. However, these examples are not intended to limit the scope of the invention.

### EXAMPLES

### Embodiment 1

### Production Example

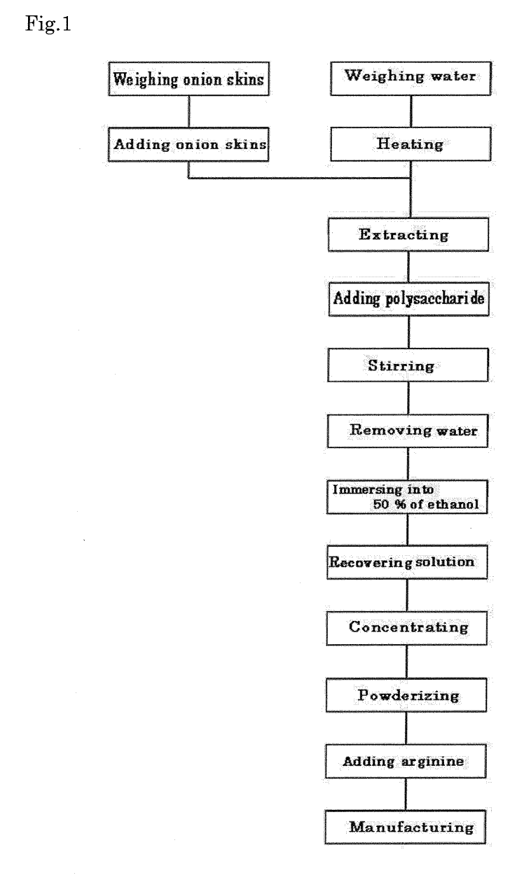

FIG. 1 is a chart showing a preferred embodiment process for purification of quercetin, which is a type of polyphenol, from onion skins. A composition for inhibiting hypertension can be obtained by adding arginine to the quercetin.

Specifically, 100 g of washed onion skins were put into 10 liters of water heated to 80° C. to extract quercetin, and impurities were removed to obtain a quercetin-containing solution.

Then, 100 g of crystalline cellulose as polysaccharide was added to the quercetin-containing solution and stirred so that quercetin is supported with the crystalline cellulose. Subsequently, water was removed and crystalline cellulose-supported quercetin was obtained.

The crystalline cellulose-supported quercetin was immersed into 10 liters of 50% ethanol solution to elute quercetin.

After eluting quercetin, purified-quercetin solution was obtained by concentrating and removing the solvent. The quercetin was powderized by freeze drying, and yielded 2 g of quercetin powder.

In addition, commercially available arginine powder was added to the quercetin powder to provide a composition for inhibiting hypertension in accordance with the present invention.

### Abilities of Various Types of Polysaccharides for Supporting Polyphenols

According to the above-mentioned production example, the ability of crystalline cellulose and various types of polysaccharide for supporting quercetin were studied. A rate of supported quercetin, which is a ratio of an amount of quercetin supported with a polysaccharide with respect to an amount of extracted quercetin, in each polysaccharide is shown in Table 1, below. A quercetin determination was measured by using liquid chromatography.

Measurement of Amount of Polyphenols ml of sample extracted in a solvent of 3 ml of methanol, 3 ml of 2 mM of diethyldithiocarbamate/methanol solution, and 2.5 ml of 50% hydrochloric acid were prepared in respective test tubes for heating 90° C. for 2 hours. After heating, 1 ml of each prepared sample was batched off, added with 2.5 ml of water, and filtered with a PTFE filter having a pore size of 0.45 μm of pore size to conduct a liquid chromatography measurement.

TABLE 1

| Support for Polyphenols | Rate of Supported Quercetin (%) |
|---|---|
| Crystalline cellulose | 95 |
| Carboxymethylcellulose | 94 |
| Cornstarch | 57 |
| Tapioca starch | 69 |
| Chitin | 65 |
| Chitosan | 64 |

Every polysaccharide which is a support for polyphenol of the present invention showed a high ability for supporting polyphenol. Crystalline cellulose and carboxymethylcellulose achieved significantly high rates of supported quercetin. Since it is possible to consume polyphenols together with polyphenol-supporting polysaccharides in the present invention, it will be appreciated that a consuming efficiency of the polyphenol supported with polysaccharide is higher than that of the polyphenol supported with conventional adsorbents. In particular, since digestible polysaccharides such as potato starch, cornstarch and tapioca starch can be digested and adsorbed in the body, it will be understood that an adsorbing efficiency of polyphenols supported with such polysaccharides is higher than that of polyphenols supported with non-digestible polysaccharides. Thus, digestible polysaccharides are preferably used as polyphenol-supporting polysaccharides which can be added to foods.

### Solvent Concentration for Supporting Polyphenols

According to the above-mentioned production example, the solvents for crystalline cellulose supporting quercetin were studied. Each concentration of ethanol, methanol and acetone instead of water in the above-mentioned production example were used as extraction solvents to obtain each quercetin-containing solution, and crystalline cellulose was added to each solvent. The subsequent operations followed the operations in the production example. Each solvent was evaluated by a rate of quercetin supported with crystalline cellulose, which is a ratio of an amount of quercetin supported with crystalline cellulose with respect to an amount of extracted quercetin. In similar fashion as previously noted, the amount of polyphenols was measured with liquid chromatography. The rates of supported quercetin in each concentration of the respective solvents are shown in Table 2, below.

TABLE 2
Rate of Supported Quercetin (%)

| Solvent Concentration | Ethanol | Methanol | Acetone |
|---|---|---|---|
| 0% (Water) | 95 | 95 | 95 |
| 10% | 62 | 53 | 50 |
| 20% | 48 | 38 | 31 |
| 30% | 22 | 29 | 10 |
| 40% | 15 | 34 | 7 |
| 50% | 8 | 14 | 3 |
| 60% | 8 | 15 | 4 |
| 70% | 5 | 5 | 4 |
| 80% | 6 | 5 | 4 |
| 90% | 2 | 4 | 4 |
| 100% | 4 | 3 | 5 |

According to Table 2, when the concentration was 40% or less, every organic solvent had a favorable rate of supported quercetin. However, when the concentration exceeded 40%, the rate of supported quercetin tended to decrease significantly as the concentration increased. Even in the case of using water as solvent, a satisfactory rate of supported quercetin can be achieved.

Thus, it is preferred that water or 40% or less concentration of ethanol, methanol or acetone solution is preferably used as solvent for adsorbing polyphenols.

### Solvent Concentration for Extracting Polyphenols

According to the above-mentioned production example, various solutions of ethanol, methanol and acetone instead of water were used as solvents for extracting quercetin from onion skins, and the amount of extracted quercetin was studied. As previously noted, the quercetin determination was measured with liquid chromatography.

The amounts of extracted quercetin in each concentration of the respective solvents are shown in Table 3, below.

TABLE 3
Amount of extracted Quercetin (mg/100 g of onion skins)

| Solvent Concentration | Ethanol | Methanol | Acetone |
|---|---|---|---|
| 0% (Water) | 1188 | 1188 | 1188 |
| 10% | 1028 | 918 | 1223 |
| 20% | 1312 | 955 | 1642 |
| 30% | 1898 | 1388 | 1692 |
| 40% | 2095 | 1108 | 1268 |
| 50% | 2383 | 1370 | 1365 |
| 60% | 2046 | 1600 | 1179 |
| 70% | 2303 | 1472 | 1358 |
| 80% | 1708 | 1411 | 1339 |
| 90% | 1553 | 1417 | 1111 |
| 100% | 1280 | 1597 | 602 |

Quercetin was extracted in every noted concentration of ethanol, methanol and acetone solution. A solution of 30 to 80% of ethanol contained a large amount of extracted quercetin. Even in the case of using water as solvent, a satisfactory amount of quercetin can be extracted.

Thus, a solution of 30 to 80% ethanol is preferably used as solvent for extracting relatively large amounts of polyphenols from polyphenol-containing materials. However, considering that a preferred solvent for supporting polyphenols is water or 40% or less of ethanol, methanol or acetone aqueous solution as described in the above-mentioned study of solvents for supporting polyphenols; if the same solvent is used in both extracting and supporting polyphenols, there is no need to change solvents in the processes, which is desirable in terms of operating efficiency and reducing costs.

### Solvent Concentration for Eluting Polyphenols

According to the above-mentioned production example, solvent concentrations for eluting quercetin from crystalline cellulose-supported quercetin were studied. Specifically, water or various concentrations of ethanol, methanol or acetone solution were used instead of 50% ethanol solution in the above-mentioned production example. That is, water or the noted solutions were used as elution solvents to elute quercetin from crystalline cellulose-supported quercetin. Each solvent was evaluated by a rate of eluted quercetin, which is a ratio of an amount of quercetin eluted into the solvent with respect to an amount of supported quercetin. As previously reported above, the quercetin determination was measured with liquid chromatography.

The rates of eluted quercetin in each concentration of the respective solvents are shown in Table 4, below.

TABLE 4
Amount of Eluted Quercetin (%)

| Solvent Concentration | Ethanol | Methanol | Acetone |
|---|---|---|---|
| 0% (Water) | 15 | 15 | 15 |
| 10% | 29 | 19 | 32 |
| 20% | 48 | 19 | 45 |
| 30% | 83 | 44 | 61 |
| 40% | 93 | 72 | 82 |
| 50% | 94 | 77 | 80 |
| 60% | 96 | 78 | 92 |
| 70% | 99 | 84 | 87 |
| 80% | 91 | 88 | 91 |
| 90% | 76 | 82 | 77 |
| 100% | 40 | 81 | 66 |

Quercetin could be eluted from every solvent of ethanol, methanol and acetone. A solution of 40 to 80% ethanol had a significantly large amount of eluted quercetin. In every solvent, when the concentration was less than 40%, the rate of eluted polyphenols tended to decrease.

Thus, a solution of 40 to 80% ethanol is preferably used as solvent for eluting polyphenols from polysaccharide-supported polyphenols.

### Rate of Recovered Polyphenols

Subsequently, polyphenols suitable for the methods of the present invention were studied.

Specifically, in the above-mentioned production example, tea, grape pomace, and red bean soup instead of onion skins were used as polyphenol-containing materials, and polyphenols contained in the respective materials were purified. Each rate of polyphenol recovered from the respective materials, which is a ratio of an amount of purified polyphenols with respect to an amount of polyphenols in a polyphenol-containing solution, is respectively shown in Table 5, below.

The red bean soup is a polyphenol-containing solution prepared by decocting 100 g of red beans with 400 ml of water to obtain its decoction. The other polyphenol-containing solutions were respectively extracted from 1 g of tea leaves, 1 g of onion skins, and 10 g of grape pomace with 100 ml of hot water each. The subsequent operations followed the operations in the production example.

TABLE 5

| Polyphenols | Rate of Recovered Polyphenols |
|---|---|
| Tea | |
| Epigallocatechin (EGC) | 9 |
| Epigallocatechin gallate (EGCg) | 29 |
| Epicatechin (EC) | 8 |
| Epicatechin gallate (ECg) | 21 |
| Grape Pomace | |
| Total Polyphenols | 11 |
| Proanthocyanidin | 18 |
| Red Bean Soup | |
| Total Polyphenols | 10 |
| Proanthocyanidin | 10 |
| Onion Skins | |
| Quercetin | 80 |

As shown in Table 5, it is noted that the rate of recovered quercetin was extremely high. In addition, the other polyphenols could be recovered stably.

According to the above-mentioned results, it was found that the preferred polyphenol used in the present invention is quercetin.

### Rate of Recovered Polyphenols 2

Furthermore, according to the above-mentioned production example, a rate of recovered quercetin when using lignocellulose as the polysaccharide to support polyphenols was studied and compared with the rate of recovered quercetin when using crystalline cellulose in the above-mentioned test. As in the above-mentioned test, polyphenol-containing solution was extracted from 1 g of onion skins with 100 ml of hot water. The other operations followed the operations in the production example. A rate of recovered polyphenol, which is a ratio of an amount of purified polyphenols with respect to an amount of polyphenols in a polyphenol-containing solution, in each cellulose is shown in Table 6, below. A quercetin determination was measured with liquid chromatography, and lignocellulose prepared with the following method was used.

### Method for Preparing Lignocellulose

100 g of cedar wood powder was dried at 50° C. for a day and then filtered with an 80 mesh screen. The obtained dried wood powder was immersed into 1 liter of 0.1 N of sodium hydroxide solution for 3 days and then washed adequately with water. Then, the powder was immersed into 1 liter of 0.1% of hydrochloric acid solution for a day and then washed adequately with water. Subsequently, the powder was immersed into 60% of ethanol solution for a day, washed adequately with water, and then dried naturally.

TABLE 6
Rate of Recovered Polyphenols (%)

| Polyphenols | | |
|---|---|---|
| Onion skins | Crystalline cellulose | Lignocellulose |
| Quercetin | 80 | 37 |

As shown in Table 6, the ability of lignocellulose for supporting quercetin was lower than that of crystalline cellulose. Thus, it became apparent that the ability of crystalline cellulose for supporting quercetin was significantly more superior than that of lignocellulose.

Lignocellulose is a cellulose containing lignin, which is copolymer existing in xylem, also known as wood powder. Crystalline cellulose is obtained by purifying only pure cellulose which is crystalline, and does not contain lignin.

In view of the above, especially in terms of purifying quercetin, it is preferred that highly pure cellulose without lignin is preferably used for supporting polyphenols.

### Example 2

### Test for Inhibiting Hypertension in Rats

A composition obtained by adding arginine to quercetin for inhibiting hypertension according to the present invention was studied.

### Preparation of Quercetin

Quercetin was extracted by adding 100 g of washed onion skins to 10 liters of water heated to 80° C., and impurities were removed to obtain quercetin-containing solution. The amount of quercetin contained in the quercetin-containing solution was 0.1 mg/ml. The quercetin-containing solution was appropriately concentrated with an evaporator.

### Test Method

11 week old male spontaneously hypertensive rats (SHR) were divided into A to I groups (9 groups consisting of 5 rats per group) and 11 week old male normotensive Wistar Kyoto (WKY) rats were divided into J to L groups (3 groups consisting of 5 rats per group) so that averages of weight and blood pressure (BP) of the respective groups were nearly equal. They were pre-bred in each breeding cage at 22.0±1.0° C. temperature, 60±10% of humidity and 12 hour light-dark cycles for a week while taking feeds and water ad libitum.

After the pre-breeding period, they were bred with the same conditions for 8 weeks. The quercetin-containing solution obtained in the above-mentioned preparation method and commercially-available arginine powder (produced by Ajinomoto Co., Inc.) were blended together so that amounts of quercetin and arginine would be the respective amounts in Table 7, below. Each mixture was orally administered to the rats in each group once a day. The rats took feed and water ad libitum during the test.

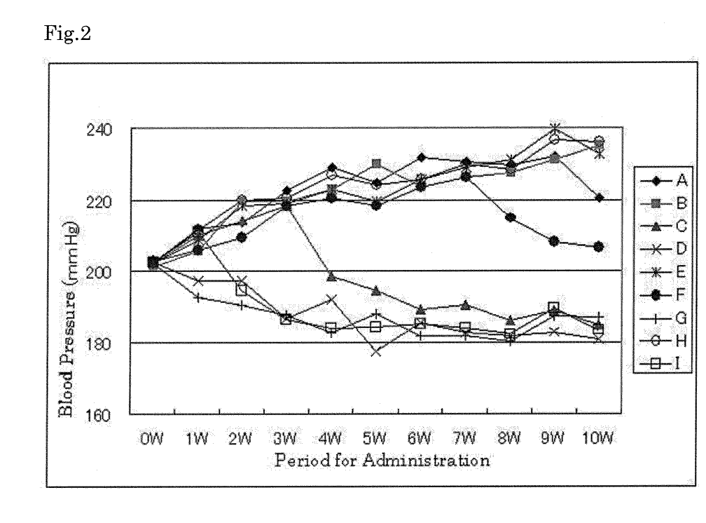

The blood pressure (BP) of rats was measured once a week. After each rat was warmed and climatized itself in a warmer at 38° C. for a few minutes, a systolic BP at the tail pulse was measured with an automatic sphygmomanometer (BP-98A; Softron Co., Ltd.) In each rat, the BP was measured for three times per one measurement and an average of the three measured values was recorded. The changes in BP in the respective groups are shown in FIG. 2.

TABLE 7

| | | Quercetin (mg/day/kg) | Arginine (mg/day/kg) |
|---|---|---|---|
| Group A | SHR | 0 | 0 |
| Group B | SHR | 0.3 | 0 |
| Group C | SHR | 0.7 | 0 |
| Group D | SHR | 2.7 | 0 |
| Group E | SHR | 0 | 0.03 |
| Group F | SHR | 0.3 | 0.03 |
| Group G | SHR | 0.7 | 0.03 |
| Group H | SHR | 0 | 0.15 |
| Group I | SHR | 2.7 | 0.15 |
| Group J | WKY | 0 | 0 |
| Group K | WKY | 0.7 | 0.15 |
| Group L | WKY | 2.7 | 0.15 |

According to FIG. 2, an increase in BP with time was observed in Group A in which quercetin and arginine were not administered, however, hypertensions were inhibited in the other groups. The groups (Group F, G and I) in which the mixture of quercetin and arginine was administered exhibit greater beneficial effect in which hypertension was inhibited compared with the groups in which either quercetin or arginine was administered.

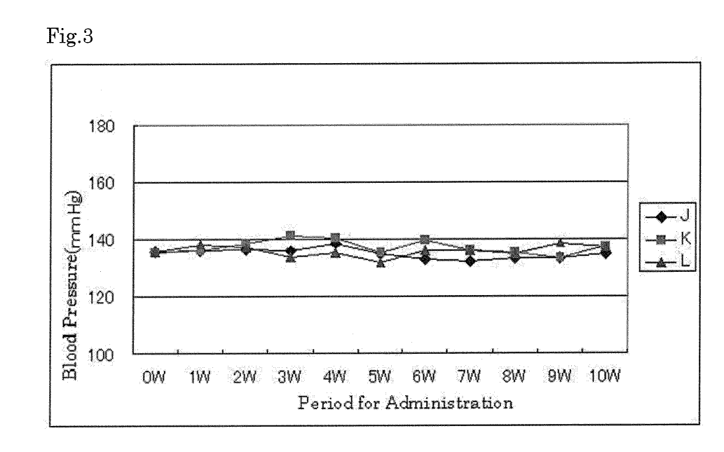

Considering the above-mentioned results and a relationship between an effect and dosage, it is understandable that favorable effects can be achieved when quercetin and arginine are blended in the ratios of 10 to 23:1. In addition, as shown in FIG. 3, the administration of quercetin and arginine did not affect the BPs of the normotensive WHY rats.

Furthermore, when using purified quercetin instead of the quercetin-containing solution in the above-mentioned test, a similar result can be achieved. Thus, it will be understood that, considering that the various foods and drinks with which the composition may be blended, taste good and do not contain toxic substances such as agrichemicals, purified quercetin is more preferably used as quercetin to be blended into the composition for inhibiting hypertension in accordance with the present invention.

Formulation examples of polysaccharide-supported polyphenols in foods and drinks will be hereinafter described. The present invention is not restricted by these examples.

FORMULATION 1:
Tablets

| | (mass %) |
|---|---|
| (1) Crystalline cellulose-supported quercetin | 44.0 |
| of the present invention | |
| (2) Dextrin | 22.0 |
| (3) Starch | 20.0 |
| (4) Sucrose esters | 6.0 |
| (5) Oligosaccharide | 3.0 |
| (6) Vitamin C | 5.0 |

FORMULATION 2:
Refreshing Beverages

| | (mass %) |
|---|---|
| (1) Starch-supported polyphenol | 1.0 |
| of the present invention | |
| (2) High-fructose corn syrup | 30.0 |
| (3) Emulsifier | 0.5 |
| (4) Sweetener amount | Appropriate |
| (5) Purified water | Balance |

1. A method for producing a polyphenol-rich composition comprising:
contacting a polyphenol-containing solution with a polysaccharide selected from the group consisting of cellulose, starch, chitin, and chitosan; and
supporting polyphenols with the polysaccharide.

2. The method of claim 1, wherein the cellulose is selected from the group consisting of crystalline cellulose, carboxymethylcellulose, microfibrillar cellulose, or powder cellulose.

3. The method of claim 1, wherein the polyphenol to be an object for high concentration is quercetin.

4. The method claim 1, wherein the polyphenol-containing solution is a solution containing quercetin extracted from onion skins with water and 40% or less of ethanol, methanol or acetone solution.

5. A food or beverage product including polysaccharide-supported polyphenols.

6. The food or beverage product of claim 5, wherein the polysaccharide is starch.

7. A method for purifying polyphenols comprising:
contacting polysaccharide-supported polyphenol with solvent to elute polyphenols into the solvent; and
powderizing the polyphenols after removing the solvent.

8. The method of claim 7, wherein the polyphenol is quercetin.

9. A composition for inhibiting hypertension comprising quercetin and arginine.

10. The method of claim 2, wherein the polyphenol to be an object for high concentration is quercetin.

11. The method of claim 2, wherein the polyphenol-containing solution is a solution containing quercetin extracted from onion skins with water and 40% or less of ethanol, methanol or acetone solution.

12. The method of claim 3, wherein the polyphenol-containing solution is a solution containing quercetin extracted from onion skins with water and 40% or less of ethanol, methanol or acetone solution.
